(19) United States
(12) Patent Application Publication
(10) Pub. No.: US 2024/0416859 A1
(43) Pub. Date: Dec. 19, 2024

(54) OMNI-WHEEL CABLE PUSHER

(71) Applicant: GLOBUS MEDICAL, INC., AUDUBON, PA (US)

(72) Inventors: Kevin Zhang, Medford, MA (US); Steven Tracy, Litchfield, NH (US); David Toombs, Hollis, NH (US); Gary Pinard, Manchester, NH (US); Norbert Johnson, North Andover, MA (US)

(21) Appl. No.: 18/821,296

(22) Filed: Aug. 30, 2024

Related U.S. Application Data
(63) Continuation of application No. 17/655,370, filed on Mar. 18, 2022, now Pat. No. 12,103,480.

(51) Int. Cl. B60R 19/54; A61B 6/00; B60R 19/00; B60S 9/14; B62B 5/00

(52) U.S. Cl. CPC B60R 19/54; A61B 6/4405; B60S 9/14; B62B 5/0086; B60B 2200/26; B60R 2019/002

(57) ABSTRACT

Cable protector devices, assemblies, and systems. The cable protection system may include a moveable system with wheels for moving the moveable system across a floor and a cable pusher attached to each of the wheels. Each cable pusher may include a frame with a pair of deflector legs configured to be positioned in front of and behind each wheel in close proximity to the floor. The cable pusher may be magnetically secured to the wheel, and the deflector legs may magnetically connect to the frame. As the wheels spin or rotate, the frame and deflector legs remain stationary, thereby pushing obstacles out of the way.

### CROSS-REFERENCE TO RELATED APPLICATIONS

This patent application is a continuation application of U.S. patent application Ser. No. 17/655,370 filed on Mar. 18, 2022, which is incorporated in its entirety herein.

### FIELD OF THE INVENTION

The present application relates generally to cable protection systems, and more particularly, to cable pushers configured to protect cables lying on the floor from damage by wheels of a moveable system.

### BACKGROUND OF THE INVENTION

The healthcare industry utilizes a number of mobile systems for patient care. In particular, mobile three-dimensional imaging systems, robotic systems, and/or navigation systems may be used in operating rooms, procedure rooms, intensive care units, emergency departments, ambulatory surgery centers, physician offices, or the like. These moveable systems may be very useful during surgery or other procedures.

The imaging, robotic, and/or navigation systems may be moveable via a set of wheels. In some cases, omni-directional wheels may be used to allow for multiple-axis movement of the system. Omni-directional wheels provide for all of the desired degrees of freedom similar to conventional casters but without swiveling. However, the moveable nature of the equipment can pose the problem of potentially damaging nearby cables on the floor. While moving the equipment, the wheels may inadvertently run over cables or leads from various angles including laterally, and the wheels may cause damage to the cables or leads. Accordingly, there is a need to protect various cables and leads lying on the floor from this type of damage.

### SUMMARY OF THE INVENTION

To meet this and other needs, cable protection devices, assemblies and systems, moveable systems with cable protectors on the wheels, and methods of installing and using the same are provided. In particular, the cable protection system may utilize features on three of the four sides of each wheel configured to contact and push nearby cables while not allowing them to wander under the wheel. The cable protector device is adaptable to omni-type wheels that provide for multiple-axis movement of the system. The cable protection system protects the wheels from various cables and leads lying flat or slightly raised from the floor that could migrate under the equipment. The cable protection system may push the cables, leads, or wires out of the way, thereby preventing any damage.

According to one embodiment, a cable protection system includes a moveable system with a plurality of wheels for moving the moveable system across a floor, and a cable pusher attached to each of the plurality of wheels. Each cable pusher may include a frame with a pair of deflector legs configured to be positioned in front of and behind each wheel in close proximity to the floor. The cable pusher may be magnetically secured to the wheel. The deflector legs may magnetically connect to the frame. As the wheels spin or rotate, the frame and deflector legs remain stationary, thereby pushing obstacles (such as cables, wires, or leads) out of the way.

The cable protection system may include one or more of the following features. Each deflector leg may include an elongate leg and a shoulder configured to mate with the frame. The shoulder may transition to the leg with a bent portion that positions the leg transverse to the shoulder and the frame when connected thereto. The shoulder may be bifurcated into two branches by a window, and magnets may be positioned in the two branches of the shoulder. The pair of deflector legs may be aligned in parallel to one another. The frame may include an upper hub configured to engage with a wheel hub of the wheel, a pair of angled arms extending distally, and a base connecting the angled arms. The upper hub of the frame may house a spinning magnet constrained inside a magnet retainer such that the spinning magnet attracts to a magnet on the wheel. The cable pusher may be attached to the wheel with a mount including a revolving central shaft and a pair of blades positioned on opposite sides of the central shaft. The wheels may be omni-directional wheels configured to allow for multiple-axis movement of the moveable system. The moveable system may be medical equipment, such as an imaging system, robotic system, and/or navigation system.

According to another embodiment, a cable pusher assembly includes a frame, first and second deflector legs, and a target mount. The frame includes an upper hub, a pair of angled arms, and a base connecting the angled arms. The base includes first and second connection areas each including north and south magnets. The first and second deflector legs each include north and south magnets configured to magnetically attract to the opposite north and south magnets of the first and second connection areas, respectively, thereby securing the first and second deflector legs to the first and second connection areas of the frame. The target mount includes a revolving central shaft and a pair of blades positioned on opposite sides of the central shaft. The upper hub of the frame houses a spinning magnet constrained inside a magnet retainer.

The cable pusher assembly may include one or more of the following features. The central shaft may include an outward projection on a front face of the mount receivable in a seat defining a cavity for the projection. The target mount including the central shaft, the seat, the spinning magnet, and the magnet retainer may be coaxially aligned along a central axis. The base and the deflector legs may include one or more indicators identifying the north and south magnets such that the magnets attract when the deflector legs are correctly positioned against the frame (and repel if not aligned properly). The magnets may be disc magnets or other suitable polar magnets.

According to yet another embodiment, a cable protection system includes a moveable system, a cable pusher, and a lifting jack. The moveable system includes a plurality of wheels for moving the moveable system across a floor. The cable pusher is attachable to one or more of the plurality of wheels. The cable pusher includes a frame with a pair of deflector legs and a mount including a revolving central shaft and a pair of blades positioned on opposite sides of the central shaft. The lifting jack is attachable to one or more of the plurality of wheels. The lifting jack includes a jack mount and a dolly. The lifting jack is configured to lift the wheel off the floor and allow for movement of the moveable system using the dolly.

The cable protection system may include one or more of the following features. The central shaft of the mount may include an outward projection defining a threaded portion configured to theadedly interface with the jack mount. The mount may be securable to the wheel with nuts positioned through openings in the blades of the mount. The lifting jack may include a jacking bolt to raise and lower the wheel. The dolly may include a plate with a plurality of caster wheels. The lifting jack and dolly may allow for the moveable system to be manually maneuvered upon power loss.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a perspective view of a cable protector coupled to an omni-directional wheel of a moveable system according to one embodiment;

FIGS. 2A-2B show a moveable station and gantry for three-dimensional imaging according to one embodiment;

FIG. 3 is a perspective view of the cable protector according to one embodiment;

FIG. 4 shows the cable protector mounted to the wheel hub of the omni-wheel of the moveable system according to one embodiment;

FIG. 5 shows a partial cross-sectional view of the cable protector with the magnetically attracted mount and constrained spinning magnet according to one embodiment;

FIGS. 6A-6B show break-away magnetically-attracted deflector legs according to one embodiment;

FIG. 7 shows the target mount of the cable protector mounted to the front of the omni-wheel according to one embodiment;

FIG. 8 shows a jack mount screwed onto the target mount for supporting a dolly system according to one embodiment; and

FIG. 9 shows the omni-wheel lifted off the floor via the dolly system according to one embodiment.

### DETAILED DESCRIPTION OF THE INVENTION

Embodiments of the disclosure are generally directed to cable protection devices, assemblies, and systems. Specifically, embodiments are directed to cable protectors or pushers that prevent damage to cables, wires, or leads lying on the floor. The cable protectors may be provided on each wheel of moveable equipment to prevent the wheels from rolling over or catching the cables. The cable protectors may be especially suitable for moveable medical equipment, such as portable medical imaging systems, robotic systems, navigation systems, and the like, although it will be appreciated that the cable protectors may also be used on the wheels of moveable equipment in other applications and industries.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments and modifications thereof are contemplated as falling within the scope of this disclosure and equivalents thereto.

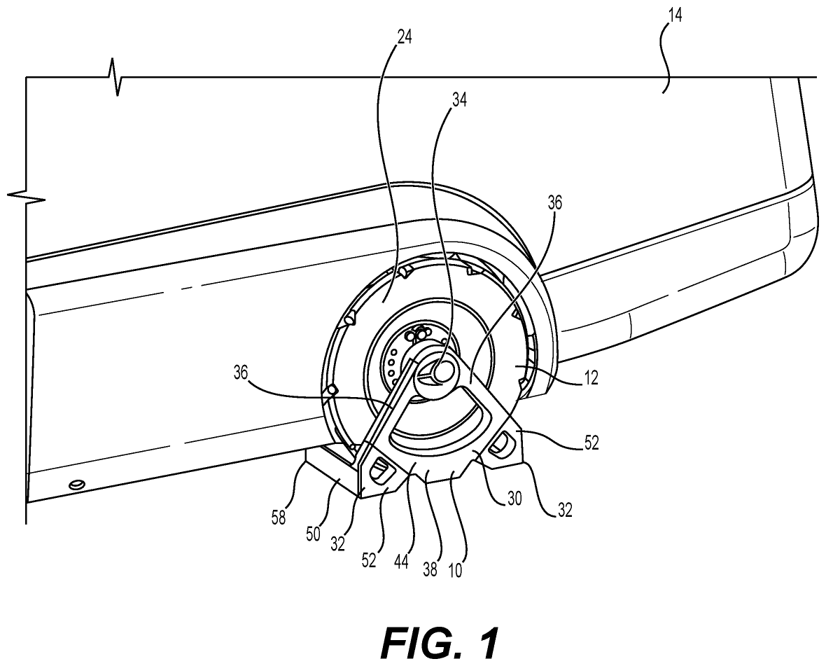

Referring now to FIG. 1, a cable protection system 10 is shown according to one embodiment. The cable protection system 10 is configured to be attached to one or more wheels 12 of moveable medical equipment 14. In one embodiment, the cable protection system 10 may be provided on each of the wheels 12 of the equipment 14. The cable protection system 10 is configured to protect any cable on or near the floor when the medical equipment 14 is moved about the room (e.g., the operating room). The cables may include any cables, wires, or leads in the room, for example, having a protective casing and used for transmitting electricity or telecommunication signals. The cable protection system 10 prevents the tangling or pinching of cables routed along the floor. During motion of the wheels 12, the cable protection system 10 comes into contact with objects first to avoid interference with the wheels 12 by pushing the obstacles out of the way. The cable protection system 10 may be configured to push or guide any cables out of the path of the wheels 12, thereby preventing the wheels 12 from rolling over the cable or the cable getting caught up into the wheels 12.

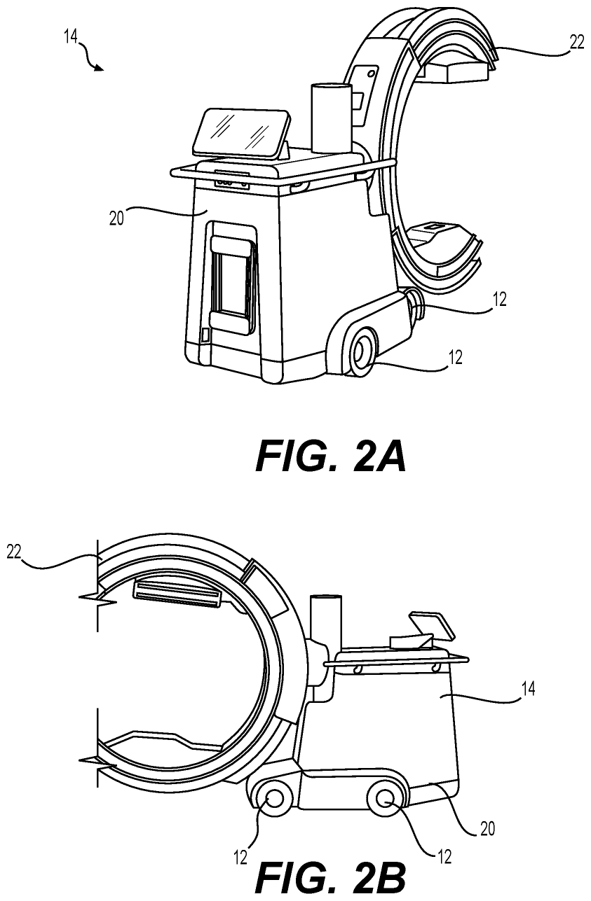

With emphasis on FIGS. 2A-2B, the medical equipment 14 may include a medical imaging system, such as a computerized tomographic (CT) x-ray scanner, including a moveable station 20 and a gantry 22. The movable station 20 may include two front wheels 12 and two rear wheels 12, which together provide movement of the movable station 20 in any direction in an X-Y plane. The gantry 22 may include a first C-arm and a second C-arm slidably coupled to the first C-arm, which are configured to provide imaging modalities including but not limited to, fluoroscopy, 2D radiography, cone-beam CT, and magnetic resonance imaging (MRI). Further details of the medical imaging system 14 is provided in U.S. Pat. No. 10,842,453, which is incorporated by reference herein in its entirety for all purposes. Although a medical imaging system is exemplified, it will be appreciated that other movable medical equipment may be provided with the cable protection systems 10 described herein.

The wheels 12 may include omni-directional wheels, which allow for the movable station 20 to be positioned in all three degrees of freedom about the horizontal plane (X, Y, Wag). The omni-directional wheels 12 allow for positioning in tight spaces, narrow corridors, or for precisely traversing up and down the length of an operating room table or patient bed. The omni-directional wheels 12 may have a central hub 24 with a plurality of smaller wheels or rollers 26 on or around its circumference. The smaller wheels 26 may be mounted at an angle to the central axis of the hub 24, thereby allowing for movement in any direction. Each wheel 12 may be powered individually to guide the portable medical imaging cart 20 in the desired direction. Further details of suitable types of omni-directional wheels 12 are provided in U.S. Pat. No. 10,842,453, which is incorporated by reference herein in its entirety for all purposes.

The medical equipment 14 is configured to move about the room via wheels 12. The equipment 14 may be moved by a person and/or with motor control. There may be cables, wire, and/or leads on or near the floor of the room, for example, from the medical equipment itself and other medical equipment used in the space. As the medical equipment 14 is moved about the space, it is desired that the wheels 12 do not run over, pinch, or otherwise damage the cables. Cable protectors or pushers 10 may be provided on each wheel 12 to prevent contact between the wheels 12 and the cables. For example, the cable pushers 10 may push or move the cables out of the way as the medical equipment 14 moves about the room.

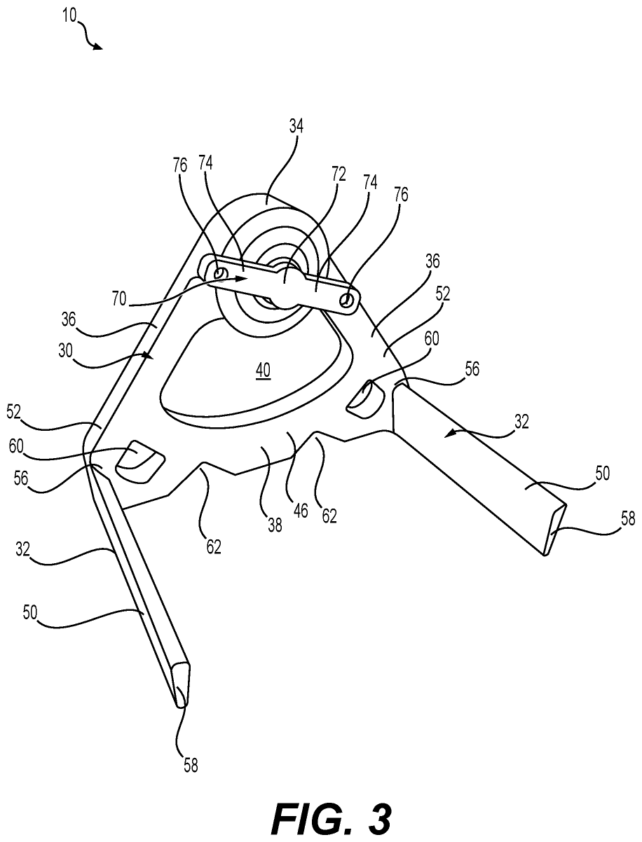

With emphasis on FIG. 3, the cable protector or pusher assembly 10 is shown in more detail. The cable protector assembly 10 includes a frame 30 with one or more deflector legs 32. The frame 30 includes an upper hub 34 configured to engage with the wheel hub 24 of the wheel 12. The outer body of the upper hub 34 may be generally rounded or curved. For example, the upper hub 34 may have a generally cylindrical body. The frame 30 may include a pair of angled arms 36 extending downwardly or distally. Each arm 36 may be angled about 20-45°, about 30-45°, or about 30° from a central longitudinal axis of the frame 30. The angled arms 36 may form a generally triangular or A-frame type configuration for the frame 30.

The distal end of each arm 36 may be connected to one another with a base 38. The frame 30 may define a cutout 40 between the hub 34, arms 36, and base 38. The cutout 40 may be generally straight along arms 36 and curved along hub 34 and base 38. The curved portion of the cutout 40 along the base 38 may be concave and the curved portion of the cutout 40 along the hub 34 may be convex. The base 38 may be segmented with a first connection portion 42 near the distal end of the first arm 36 and a second connection portion 42 near the distal end of the second arm 36. The connection portions 42 are configured to secure first and second deflector legs 32, respectively. A central portion of the base 38 between the two connection portions 42 may be configured to contact or be in close proximity to the floor.

The frame 30 has an outward-facing face 44 and an opposite inward facing face 46. The inward face 46 is configured to face toward the wheel hub 24 when the cable protector 10 is attached to the wheel 12. The outward face 44 may have a generally planar surface. The components of the frame 30 may be formed from a solid material or may be separate components that are joined together, for example, with pins, adhesive, or the like. The cable protector 10 may be formed from metals, such as steel, aluminum, or iron; plastics, such as polyurethane or polyvinylchloride; rubber; or combinations thereof.

Each deflector leg 32 includes an elongate blade or leg 50 and a shoulder 52. The shoulder 52 includes connection portion 54 configured to mate with the corresponding connections portions 42 on the frame 30. The shoulder 52 transitions to the leg 50 with an angled or bent portion 56. The bent portion 56 may be offset to one side of the shoulder 52. The bent portion 56 positions the leg 50 transverse to the shoulder 52 and body of the frame 30 when connected thereto. For example, the bent portion 56 may be angled about 80-100°, or about 90° such that the leg 50 extends generally perpendicular to the shoulder 52 and body of the frame 30 when connected thereto. Each blade or leg 50 terminates at a free end 58. When the cable protector 10 is attached to the wheel 12, the leg 50 is configured to extend back and under the wheel 12. Thus, the length of the leg 50 may be equal to or greater than the width of the wheel 12, thereby providing adequate protection to the wheel 12. With two legs 32 attached to the frame 30 (i.e., left and right deflector legs 32), the legs 50 are positioned in front of and behind the wheel 12, respectively. The pair of legs 50 may be aligned generally in parallel to one another. Each leg 50 also has a height configured to block any cables or leads lying on or near the floor. The legs 50 may be slightly angled away from one another such that the top of each leg 50 is further away from one another and the bottom of each leg 50 closer together. In this manner, the legs 50 protect the wheel 12 during travel in any direction.

To assist in assembly and/or with visibility, each shoulder 52 may define a cutout or window 60. The window 60 may bifurcate the shoulder 52 into two branches configured to receive the magnets 90, 92. The window 60 may form an arch shape with rounded corners. The connection portion 54 may form a flat face configured to mate against a corresponding flat face of the connection portion 42 on the frame 30. When the legs 32 are coupled to the frame 30, notches 62 may be defined between the shoulder 52 and the base 38 of the frame 30. The notches 62 may be generally triangular in shape. The notches 62 may be formed on the underside of the cable protector assembly 10, thereby providing clearance from the floor.

The cable pusher assembly 10 may be attached to the wheel 12 with a mount 70. The mount 70 may form a spinning target mount including a revolving central shaft 72 with one or more blades 74. The central shaft 72 may include a projection 73 on the front face of the mount 70. The projection 73 may define a circular or cylindrical interface. In one embodiment, a pair of blades 74 are aligned with one another on opposite sides of the shaft, 72. Although two blades 74 are exemplified, it will be appreciated that the mount 70 may include a suitable number of blades 74 or other geometry to secure the cable pusher assembly 10 to the wheel assembly 12. When attached to the wheel 12, the mount 70 is configured to revolve such that the mount 70 keeps pace with the revolution of the wheel 12 while holding the frame 30 and legs 32 steady.

The mount 70 may be secured to the wheel 12 with one or more fastening mechanisms. In one embodiment, the mount 70 is magnetically attracted to the wheel hub 24. In this manner, the cable pusher assembly 10 can be easily attached and removed by hand, without any tools. The mount 70 may also be attached with one or more fasteners, such as screws, bolts, or nuts 104. Openings 76 may be provided near the free end of each blade 74. The openings 76 may be configured to receive the respective fasteners, thereby allowing the mount 70 to be optionally bolted to the wheel hub 24.

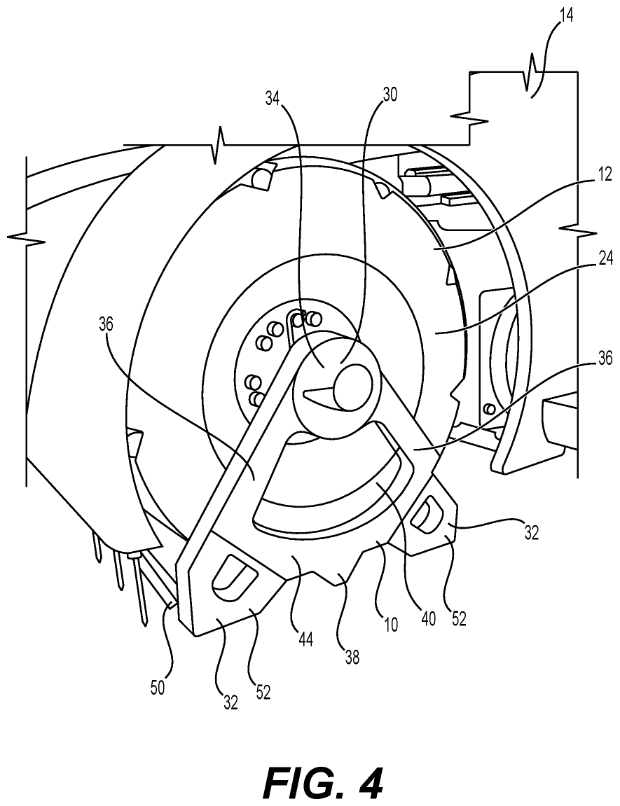

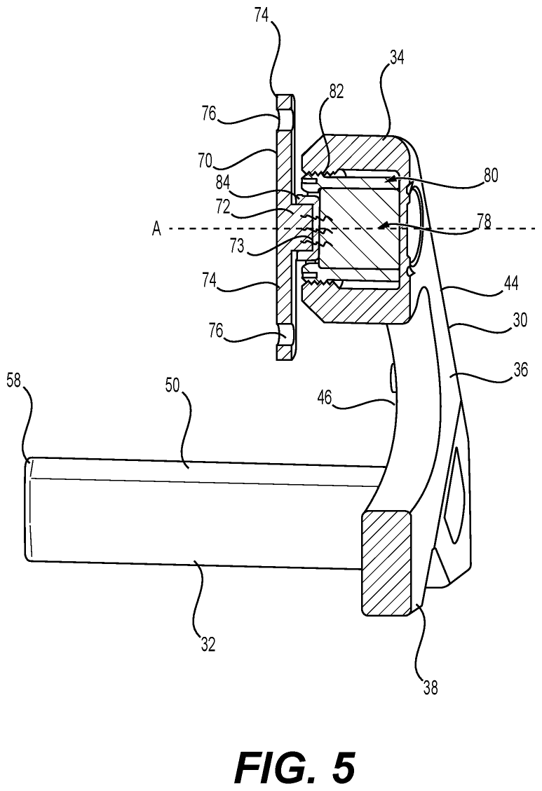

As best seen in FIG. 4, when attached to the wheel assembly 12, the mount 70 allows for the deflector legs 32 to hang slightly off the floor vertically and stationary while the wheel 12 spins. This may be accomplished using the magnetically attracted mount 70 with an internally constrained spinning magnet 78. As best seen in FIG. 5, the spinning magnet 78 may be held inside a magnet retainer 80. The internally constrained spinning magnet 78 may be positioned inside the hub 34 of the frame 30. The magnet retainer 80 may have a threaded interface 82 with the frame hub 34. The internally constrained spinning magnet 78 acts as a shaft that attracts to a ferrous target on the wheel's center hub 24.

The mount 70 may be separated from the frame 30 and magnetically attracted to the frame 30 via the constrained magnet 78. The outward projection 73 of the mount 70 is receivable in a seat 84 which defines a cavity for receiving the projection 73. The seat 84 may be sandwiched between the projection 73 and the constrained magnet 78. The mount 70 including shaft 72, seat 84, spinning magnet 78, and magnet retainer 80 may be coaxially aligned along central axis A. The spinning magnetic target mount 70 magnetically attaches to the wheel hub 24. When the assembly 10 is attached to the wheel 12, the central axis A is aligned to and corresponds with the central rotational axis of the wheel hub 24. As the wheel 12 spins or rotates, the frame 30 and deflector legs 32 remain stationary, thereby pushing any obstacles out of the way.

The cable pusher assembly 10 is configured to easily detach as a whole from each wheel 12 so that the cable pusher assemblies 10 can be quickly removed prior to traversing over a fixed or rigid obstacle, such as a threshold. In the event that the cable pusher assemblies 10 are forgotten to be removed and inadvertently run into a threshold, each deflector leg 32 may utilize a magnetic break-away interface in order to prevent serious damage to the equipment 14 as well as the cable pusher assembly themselves. Upon impact, the deflector legs 32 may detach while the main frame 30 rotates on the wheel 12. A quick reassembling and mounting gets the deflector legs 32 back to being operational.

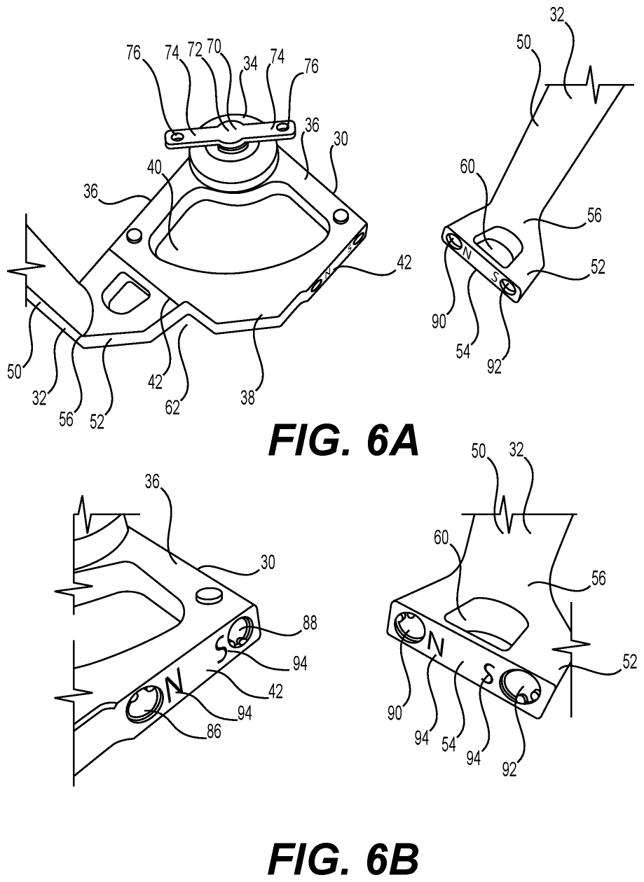

With emphasis on FIGS. 6A-6B, the deflector legs 32 couple to the frame 30 at connection portions 42, 54. Each connection portion 42, 54 may include magnetic attachment points with one or more magnets 86, 88, 90, 92. On the connection portion 42 of the frame 30 for attaching each deflector leg 32, the frame 30 may include a north magnet 86 and a south magnet 88. Similarly, on the connection portion 54 for each deflector leg 32, the shoulder 52 may include a north magnet 90 and a south magnet 92. The magnets 86, 88, 90, 92 may include polar magnets, such as cylinder magnets, disc magnets, bar magnets, rings magnets, etc. It will be appreciated that the locations of north and south magnets may be reversed, the number of magnets may be changed, and the positions of the magnets may be otherwise configured to magnetically attach the deflector legs 32 to the frame 30.

When aligned properly and placed near one another, the north pole of one magnet 86, 90 is attracted to the south pole of the opposite magnet 88, 92. The connection portions 42, 54 may include one or more indictors 94 to identify the polarity of the magnets 86, 88, 90, 92 and/or the proper positioning of the deflector legs 32 relative to the frame 30. In other words, the north and south magnetic attachment points may be arranged and marked in such a way that correct positioning of the left and right deflector legs 32 occur by attracting when correct and repelling when incorrect.

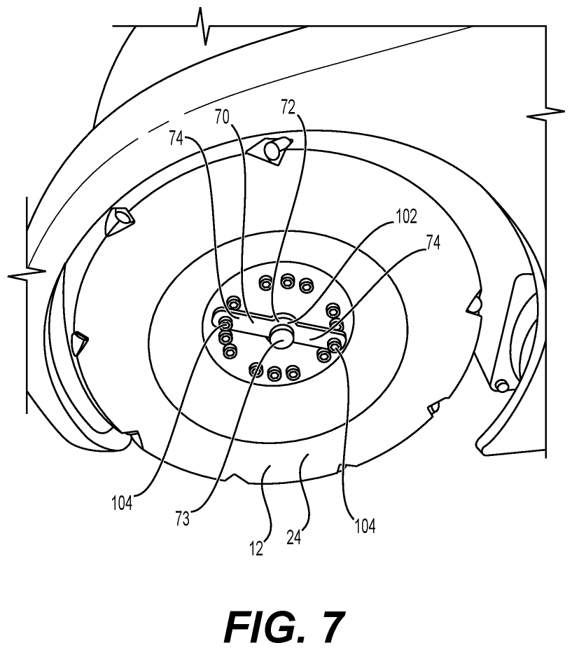

Referring now to FIGS. 7-9, the cable pusher target mount 70 allows for attachment of a lifting jack 100 in addition to the cable pushers 10. The target mount 70 that supports the cable pusher assembly 10 magnetically via its front face may also include a threaded interface 102 that can support additional attachments, such as lifting jack 100. As best seen in FIG. 7, the projection 73 on the target mount 70 may include a threaded section 102 with one or more threads around a periphery of the projection 73 configured to mate with corresponding threads on the jack 100. The mount 70 is attached to the wheel hub 24 with nuts 104 secured through openings 76 in the blades 74 of the mount 70.

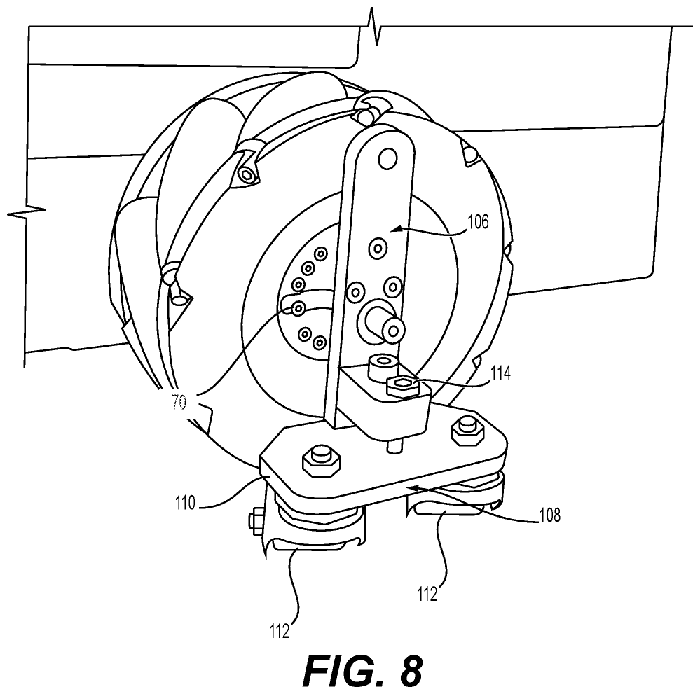

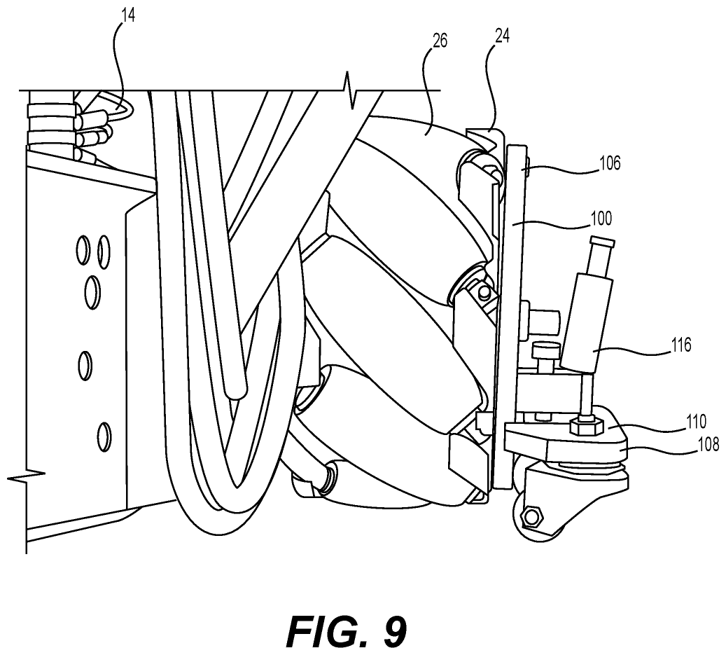

With further emphasis on FIG. 8, the lifting jack 100 may be secured to the target mount 70 via the threaded interface. The lifting jack 100 may include a jack mount 106, which is shown screwed onto the target mount 70, and a dolly 108. The dolly 108 may include a plate 110 with a plurality of wheels 112 bolted to the plate 110. The dolly wheels 112 may include a pair of caster wheels, for example. The lifting jack 100 may utilize a jacking bolt 114 to raise and lower the wheel 12. As shown in FIG. 9, an instrument 116, such as a socket wrench, may be used to turn the jacking bolt 114, thereby raising wheel 112. As shown in FIG. 9, the omni-wheel 12 is lifted off the floor, thereby allowing for movement of the equipment 14 using the dolly 108. This may be especially helpful in the event of power loss of the moveable system 14. Due to its large mass and the type of omni-wheels 12, the system 14 may be difficult to move manually. By jacking up the two rear wheels 12, for example, via the lifting jack 100 and wheel dolly 108 system, the movable system 14 is more easily maneuvered manually upon power loss. The lifting jack 100 and wheel dolly 108 may assist in the manual movement of the moveable system 14.

The cable protection assembly is configured to protect cables lying on or near the floor from damage by wheels of a moveable system, such as an imaging system. The cable protectors may be especially suitable for use with omni-type wheels that provide for multiple-axis movement of the moveable system. The cable protectors eliminate the need to reach under the wheel to assemble the assembly. Therefore, these cable protectors are less dangerous than snap-together sliding floor ring type protectors used for standard swivel casters. The cable protectors greatly reduce opportunities for breakage of the protectors even upon sudden contact due to the break-away magnetic deflector legs. Minimal sliding contact on the wheels and floor greatly reduce wear and noise since the assemblies hang a slight distance from the floor. Due to the magnetic interface, no tools are required for mounting the cable protectors to the wheels or reassembling after a collision. The cable pusher mount also provides an additional interface for other attachments, such as a wheel jacks.

Although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all components of the various devices disclosed above may be combined or modified in any suitable configuration.

What is claimed is:

1. A cable protection system comprising:
a moveable system with a plurality of wheels for moving the moveable system across a floor; and
a cable pusher attached to each of the plurality of wheels, each cable pusher comprising a frame with a pair of deflector legs configured to be positioned in front of and behind each wheel in close proximity to the floor, wherein the cable pusher is magnetically secured to the wheel, and the deflector legs magnetically connect to the frame.

2. The system of claim 1, wherein each deflector leg includes an elongate leg and a shoulder configured to mate with the frame.

3. The system of claim 2, wherein the shoulder transitions to the leg with a bent portion that positions the leg transverse to the shoulder and the frame when connected thereto.

4. The system of claim 2, wherein the shoulder is bifurcated into two branches by a window, and magnets are positioned in the two branches of the shoulder.

5. The system of claim 1, wherein the pair of deflector legs are aligned in parallel to one another.

6. The system of claim 1, wherein the frame includes an upper hub configured to engage with a wheel hub of the wheel, a pair of angled arms extending distally, and a base connecting the angled arms.

7. The system of claim 6, wherein the upper hub of the frame houses a spinning magnet constrained inside a magnet retainer, wherein the spinning magnet attracts to a magnet on the wheel.

8. The system of claim 1, wherein the cable pusher is attached to the wheel with a mount including a revolving central shaft and a pair of blades positioned on opposite sides of the central shaft.

9. The system of claim 1, wherein the wheels are omni-directional wheels.

10. The system of claim 1, wherein the moveable system is a medical imaging system.

11. A cable pusher assembly comprising:
a frame including an upper hub, a pair of angled arms, and a base connecting the angled arms, the base including first and second connection areas each including north and south magnets;
first and second deflector legs each including north and south magnets are configured to magnetically attract to the opposite north and south magnets of the first and second connection areas; and
a target mount including a revolving central shaft and a pair of blades positioned on opposite sides of the central shaft, wherein the upper hub of the frame houses a spinning magnet constrained inside a magnet retainer.

12. The cable pusher assembly of claim 11, wherein the central shaft includes an outward projection on a front face of the mount, and the projection is received in a seat defining a cavity for the projection.

13. The cable pusher assembly of claim 12, wherein the target mount including the central shaft, the seat, the spinning magnet, and the magnet retainer are coaxially aligned along a central axis.

14. The cable pusher assembly of claim 11, wherein the base and the deflector legs include indicators identifying the north and south magnets such that when the deflector legs are correctly positioned against the frame the magnets attract.

15. The cable pusher assembly of claim 11, wherein the magnets are disc magnets.

16. A cable protection system comprising:
a moveable system with a plurality of wheels for moving the moveable system across a floor;
a cable pusher attachable to one or more of the plurality of wheels, the cable pusher comprising a frame with a pair of deflector legs and a mount including a revolving central shaft and a pair of blades positioned on opposite sides of the central shaft; and
a lifting jack attachable to one or more of the plurality of wheels, the lifting jack including a jack mount and a dolly.

17. The system of claim 16, wherein the central shaft of the mount includes an outward projection defining a threaded portion configured to theadedly interface with the jack mount.

18. The system of claim 16, wherein the mount is securable to the wheel with nuts positioned through openings in the blades of the mount.

19. The system of claim 16, wherein the lifting jack includes a jacking bolt to raise and lower the wheel.

20. The system of claim 16, wherein the dolly includes a plate with a plurality of caster wheels.
